(19) United States
(12) Patent Application Publication
(10) Pub. No.: US 2022/0408758 A1
(43) Pub. Date: Dec. 29, 2022

(54) DRIED KUDZU FLOWER POWDER AND FOOD PRODUCT COMPRISING DRIED KUDZU FLOWER POWDER

(71) Applicant: TOYO SHINYAKU CO., LTD., Fukuoka (JP)

(72) Inventors: Kohei FUJIKI, Saga (JP); Hideki UEDA, Saga (JP); Aki MIYAMOTO, Saga (JP); Chisato HOTTA, Saga (JP); Tsuyoshi SATO, Saga (JP); Tomoyasu KAMIYA, Saga (JP)

(73) Assignee: TOYO SHINYAKU CO., LTD., Fukuoka (JP)

(21) Appl. No.: 17/642,033

(22) PCT Filed: Sep. 11, 2020
(86) PCT No.: PCT/JP2020/034390

§ 371 Date: Mar. 10, 2022

(30) Foreign Application Priority Data
Sep. 12, 2019 (JP) 2019-166687

(51) Int. Cl. A23L 2/39; A23L 2/38; A23L 33/10; A23L 5/00

(52) U.S. Cl. CPC A23L 2/39; A23L 2/38; A23L 33/10; A23L 5/00; A23V 2300/10; A23V 2002/00

(57) ABSTRACT

The present invention is a food product comprising dried Kudzu flower powder, and having a water content of 8% by weight or less, and preferably which proportion of fractions passing a sieve having a sieve opening of 2 mm is 20% by weight or more. The food product of the present invention has good taste and odor.

### TECHNICAL FIELD

The present invention relates to dried Kudzu flower powder and food product comprising the same.

### BACKGROUND ART

Kudzu is a plant of pea family having a long trailing vine, and Kudzu starch collected from its root has been used as a raw material of Japanese cakes since early times. Further its root and flower are each called “Puerariae radix” and “Kudzu flower”, respectively, and have been employed since early times in China, as being effective as antifebrile, antidiarrheic, or for alcoholic intoxication, or the like.

As Kudzu flower, Kudzu flower extract (Kudzu flower essence) extracted by using a solvent is widely used. Those using Kudzu flower extract, for example, a food composition for preventing or ameliorating menopause symptoms comprising Kudzu flower extract, and at least one extract selected from the group consisting of Citri Fructus Aurantii and Citri Pericarpium (Patent document 1).

### CITATION LIST

### Patent Literature

Patent document 1: Japanese Laid Open Patent Publication No. 2017-197519

### SUMMARY OF INVENTION

### Technical Problem

However, Kudzu flower extract had problems since it requires extraction treatment, increases manufacturing steps making the manufacture complex. Further, since Kudzu flower extract has a unique flavor (taste and odor), it could not be said to be eaten as it is, or suitable to mix in a wide variety of foods.

The object of the present invention is to provide a dried Kudzu flower powder suitable as a food product having a good flavor, and a food product comprising the same.

### Solution to Problem

The present inventors made a keen study on food products using Kudzu flower, and focused on using dried Kudzu flower powder, to ameliorate the unique flavor of Kudzu flower extract. Specifically, they found out that the dried Kudzu flower powder had a good flavor as a food product, as compared to the Kudzu flower extract. The present inventors further made a keen study on the dried Kudzu flower powder, and as a result they found out by adjusting the water content of the dried Kudzu flower powder at a predetermined amount, its good flavor can be maintained over a long period of time. The present invention has been thus completed.

Specifically, the present invention is as follows.
[1] A dried Kudzu flower powder, having a water content of 8% by weight or less.
[2] The dried Kudzu flower powder according to [1], wherein a proportion of fractions passing a sieve having a sieve opening of 2 mm is 20% by weight or more.
[3] The dried Kudzu flower powder according to [1] or [2], wherein the dried Kudzu flower powder is for food use.
[4] A food product comprising the dried Kudzu flower powder according to any one of [1] to [3]
[5] A food product comprising a dried Kudzu flower powder, and having a water content of 8% by weight or less.
[6] The food product according to [4] or [5], wherein the food product is for decreasing body fat.
[7] The food product according to [4] or [5], wherein the food product is for ameliorating obesity.
[8] The food product according to [4] or [5], wherein the food product is for ameliorating cholesterol in blood.

### Advantageous Effects of Invention

The dried Kudzu flower powder and the food product containing the same of the present invention have a good taste and odor.

### DESCRIPTION OF EMBODIMENTS

The dried Kudzu flower powder and the food product containing the same of the present invention is characterized by that the water content is 8% by weight or less.

The dried Kudzu flower powder has a good taste and odor, compared to an extract of Kudzu flower and is suitable as food product.

Further, since the water content is 8% by weight or less, it has the following effects.
(1) Good taste, odor is maintained over a long period of time, and it is possible to store for a long period of time, and a sufficient best-before period can be ensured, and is suitable to use as a food product.
(2) When ingesting dried Kudzu flower powder as it is, since it has a good savor, it is suitable to use as a food product (powdered food product, tablets, etc.) ingested as it is (without suspending in water, etc.).
(3) Since it is easily dispersed in water (hot water) etc., it is also suitable to use as a powder beverage (instant beverage).
(4) Since the elution amount of tectorigenins (useful ingredient of Kudzu flower) to water (hot water) etc. is large, it is also suitable to use as a tea bag beverage.

The water content of the dried Kudzu flower powder and of the food product containing the same of the present invention is preferably 3% by weight or more. When the water content is 3% by weight or more, the cell activating activity tends to increase. In the food product of the present invention, the water content in the whole food product and the water content of the dried Kudzu flower powder being mixed are considered to be the same.

Kudzu is a trailing perennial plant in the pea family Fabaceae, and in the present invention flower of Kudzu is used. As Kudzu flower in the present invention, those collected in any state from flower bud to full bloom can be used, and those collected in each stage can be mixed and used. Species of Kudzu are not particularly limited, and examples include Pueraria thomsonii, Pueraria lobata, and Pueraria thunbergiana, etc. From the viewpoint of easiness to obtain or various effects, Pueraria thomsonii is preferable.

The dried Kudzu flower powder in the present invention is a powder obtained by drying and grounding collected Kudzu flower, and dried powder of extracted product (extract) or juice is not included. Specifically, for example, those obtained by washing, drying the collected Kudzu flower to make a dried Kudzu flower, then grounded to make a dried Kudzu flower powder can be exemplified.

As for the particle size distribution (weight basis) of the dried Kudzu flower powder in the present invention is, from the viewpoint of flavor, dispersibility, or elution of tectorigenins, etc. it is preferred that the proportion of fractions passing a sieve opening of 2 mm, preferably a sieve of 1.18 mm is 5% by weight or more, more preferably 10% by weight or more, further more preferably 20% by weight, most preferably 50% by weight or more, most further preferably 70% by weight or more, and particularly preferably 80% by weight or more. Further, the above-mentioned fractions are preferably fractions not passing a sieve opening of 0.25 mm, preferably a sieve of 0.15 mm. Specifically, for example, examples of the above-mentioned fractions include a fraction passing a sieve opening of 2 mm, and not passing a sieve opening of 0.25 mm, or a fraction passing a sieve opening of 1.18 mm, and not passing a sieve opening of 0.15 mm.

The lower limit of the proportion of fractions passing a sieve opening of 2 mm and not passing a sieve opening of 0.25 mm is not particularly limited, but from the viewpoint of flavor, dispersibility, or elution of tectorigenins, etc. it is preferred to be 1.5% by weight or more, more preferably 5% by weight or more, further more preferably 10% by weight or more, most preferably 20% by weight or more, most further preferably 50% by weight or more, and particularly preferably 65% by weight or more. The upper limit of the proportion of fractions passing a sieve opening of 2 mm and not passing a sieve opening of 0.2.5 mm is not particularly limited, but from the viewpoint of flavor, dispersibility, or elution of tectorigenins, etc. it is preferred to be 99% by weight or less, more preferably 98% by weight or less, further more preferably 95% by weight or less, most preferably 94% by weight or less, most further preferably 93% by weight or less, and particularly preferably 92% by weight or less.

Further, the lower limit of the proportion of fractions passing a sieve opening of 1.18 nun and not passing a sieve opening of 0.15 mm is not particularly limited, but from the viewpoint of flavor, dispersibility, or elution of tectorigenins, etc. it is preferred to be 5% by weight or more, more preferably 10% by weight or more, further more preferably 20% by weight or more, most preferably 40% by weight or more, most further preferably 60% by weight or more, and particularly preferably 70% by weight or more. The upper limit of the proportion of fractions passing a sieve opening of 1.18 mm and not passing a sieve opening of 0.15 mm is not particularly limited, but from the viewpoint of flavor, dispersibility, or elution of tectorigenins, etc. it is preferred to be 99.5% by weight or less, more preferably 99% by weight or less, further more preferably 98% by weight or less, most preferably 97% by weight or less, most further preferably 96% by weight or less, and particularly preferably 95% by weight or less.

Further, the upper limit of the particle diameter (median diameter (weight basis)) of the dried Kudzu flower powder in the present invention is, from the viewpoint of flavor, dispersibility, or elution of tectorigenins, etc. preferably 2 mm or less, more preferably 1.65 mm or less, particularly preferably 1 mm or less. it is preferably 0.15 to 1.65 mm, more preferably 0.18 to 1.65 mm, further more preferably 0.25 to 1.00 mm, most preferably 0.30 to 0.90 mm, most further preferably 0.35 to 0.80 mm, and particularly preferably 0.38 to 0.70 mm

The above particle size distribution and median diameter can be measured for example by using a robot shifter manufactured by Seishin Enterprise Co., Ltd.

Conventionally, in a tea bag beverage filled with plant powder such as flower tea characterized by its odor, relatively large powder was often used. Specifically, when storing after finely grounding, since odor immediately disappear during the storage period, it was used in a state that is not finely ground. On the other hand, the dried Kudzu flower powder having a water content of 8% by weight or less, even by grounding to a fine particle diameter as in the above, the deterioration speed of the odor is slow, and the odor can be kept even in fine powder.

Further, the aerated bulk density of the dried Kudzu flower powder in the present invention is, from the viewpoint of easiness of filling in case of manufacturing powder food product such as powder beverage or tea bag beverage, preferably 0.2 g/mL or more.

Further, the compression degree of the dried Kudzu flower powder in the present invention is, from the viewpoint of easiness of tilling in case of manufacturing powder food product containing dried Kudzu flower powder, such as powder beverage or tea bag beverage, preferably 11% or more. The compression degree is a value calculated from the difference between packed bulk density and aerated bulk density.

The aerate bulk density was obtained by filling dried Kudzu flower powder in a container of a predetermined volume, levelling off the powder on the top of the container, and measuring its weight. Further, the packed bulk density was obtained by filling dried Kudzu flower powder in a container of a predetermined volume, tapping for 180 times, levelling off the powder on the top of the container and measuring its weight.

As for the specific surface area (helium adsorption BET method) of the dried Kudzu flower powder in the present invention, from the viewpoint of flavor, dispersibility, or elution of tectorigenins, etc., it is preferably 0.3 m²/g or more, more preferably 0.4 m²/g or more, further more preferably 0.5 m²/g or more, most preferably 0.6 m²/g or more, most further preferably 0.65 m²/g or more, and particularly preferably 0.7 in²/g or more.

The lower limit of the repose angle of the dried Kudzu flower powder in the present invention is preferably 31 degree or more, more preferably 44 degree or more, further more preferably 45 degree or more, most preferably 46 degree or more, and particularly preferably 47 degree or more. The upper limit of the repose angle of the dried Kudzu flower powder in the present invention is preferably 65 degree or less, more preferably 55 degree or less, further more preferably 53 degree or less, most preferably 52 degree or less, further most preferably 51 degree or less, and particularly preferably 50 degree or less. The repose angle can be measured for example by using a Multitester MT1000 manufactured by Seishin Enterprise Co., Ltd.

The content of the dried Kudzu flower powder in the food product of the present invention is not particularly limited, but generally, is for example 0.01 to 100% by weight, preferably 1 to 95% by weight, more preferably 3 to 90% by weight, and most preferably 5 to 85% by weight with respect to the total amount of the food product. Further, when using as powder beverage or tea bag beverage, the content of dried Kudzu flower powder is preferably 5% by weight or more, more preferably 10% by weight or more, further more preferably 20% by weight or more, most preferably 50% by weight or more, further most preferably 80% by weight or more, and particularly preferably 90% by weight or more. Further, when using dried Kudzu flower powder as a tea bag beverage, it may be a tea bag beverage comprising only dried Kudzu flower powder.

Since dried Kudzu flower powder contains isoflavone such as tectorigenins, etc., it is effective for reducing body fat, and the food product of the present invention can be used as general food product as well as for example, so called health food products such as functional foods which indication of efficacy is allowed from a prescribed authority, including foods for specified health use, foods with nutrient function claims, foods with function claims, or the like. The content of tectorigenins in dried Kudzu flower powder is, for example, preferably 1 to 25% by weight, more preferably 3 to 20% by weight, and further more preferably 5 to 15% by weight. Tectorigenins refer to at least one compound selected from tectorigenin, tectoridin and tectorigenin 7-O-xylosylglucoside (TGXG). Further, when tectorigenins comprise 2 or more of the above compounds, the content of tectorigenins refers to the total amount of these.

Specifically, the food product of the present invention can be used as a food for ameliorating obesity (dietary food) such as food for reducing body fat. The food for ameliorating obesity is not particularly limited as long as it comprises dried Kudzu flower powder having a water content of 8% by weight or less, and can be distinguished from other products as product, in the point of being used for ameliorating obesity, and for example those with an indication of having a function related to amelioration of obesity such as function of reducing body fat on any of the main body, package, instructions, advertisement (advertising medium) of the product of the present invention are encompassed in the scope of the present invention. Further, the food for ameliorating obesity of the present invention is not limited to those with an indication of comprising Kudzu flower as active ingredients on the package of the product, etc. For example, it can be one not specifying the active ingredients. Further, even it is a general food product, those manufactured and sold by suggesting the use are encompassed in the scope of the food product for ameliorating obesity of the present invention.

Specifically, in a so-called health food, those indicating “suppressing fat absorption”, “to those getting worried about their body weight”, “aiding decreasing body weight, body fat of those getting fat”, “aiding decreasing visceral fat (fat of stomach) and BMI of those getting fat”, “to those getting worried about visceral fat”, “decreasing (ameliorating) higher BMI”, “to those getting worried about their higher IBMI”, “making easier to consume fat”, “supporting style”, or the like can be exemplified.

Further, the food product of the present invention can be used as a food for ameliorating cholesterol in blood. The food for ameliorating cholesterol in blood is not particularly limited as long as it comprises dried Kudzu flower powder having a water content of 8% by weight or less, and can be distinguished from other products as product, in the point of being used for ameliorating cholesterol in blood such as lowering cholesterol, suppressing increase of cholesterol, and for example those with an indication of having a function related to amelioration of cholesterol in blood on any of the main body, package, instructions, advertisement (advertising medium) of the product of the present invention are encompassed in the scope of the present invention. Further, the food for ameliorating cholesterol in blood of the present invention is not limited to those with an indication of comprising Kudzu flower as active ingredients on the package of the product, etc. For example, it can be one not specifying the active ingredients. Further, even it is a general food product, those manufactured and sold by suggesting the use are encompassed in the scope of the food product for ameliorating cholesterol in blood of the present invention.

Specifically, in a so-called health food, those indicating “suppressing increase of cholesterol”, “to those getting worried about cholesterol”, “lowering (ameliorating) higher cholesterol”, “lowering total cholesterol or bad cholesterol (LDL)”, or the like can be exemplified.

The amount of intake of health food of the present invention is not particularly limited. However, to further increase the action, in case it is a food product with which dried Kudzu flower powder is ingested as it is, it is preferable to intake so that the amount of intake of dried Kudzu flower powder of an adult per day is 0.1 mg or more, more preferable to intake so that it is 10 mg or more, and most preferable to intake so that it is 50 mg or more. The upper limit is for example 5000 mg, preferably 3000 mg, and more preferably 2000 mg. in case it is a food product that is ingested by extracting dried Kudzu flower powder with hot water, etc. such as tea bag beverage, it is preferable to intake so that the amount of intake of dried Kudzu flower powder (amount contained in tea bag) of an adult per day is 0.01 mg or more, more preferable to intake so that it is 1 mg or more, and most preferable to intake so that it is 100 mg or more. The upper limit is for example 5000 mg, preferably 3000 mg, and more preferably 2000 mg.

The health food product of the present invention can be appropriately designed so that the amount of intake per day becomes the above-mentioned amount of intake, and it may be taken at once, or taken in plural times. For example, it may be taken 1 to 4 times per day, and it suffices that the above amount of intake is taken as a total amount. The health food of the present invention can be stored as an amount per day in one container, or for example in two or more plural containers, so that the amount of intake per day becomes the above-mentioned amount of intake.

Examples of the form of food product of the present invention include, for example, tablet form, capsule form, powder form, granule form, grain form, bar form, plate form, block form, solid form, pellet form, caplet form, chewable tablet form, stick form, jelly form, or the like. Among these, in case of a health food, the form of tablet form, capsule form, powder form, granule form, pellet form, chewable tablet form is preferable.

In case it is in a powder form or granule form, it can be used as powder beverage or tea bag beverage. In case of a tea bag beverage, since the elution amount of tectorigenins (useful ingredients of Kudzu flower) to water (hot water) etc. of the dried Kudzu flower powder of the present invention is large, the effect of reducing body fat can be effectively obtained.

The food product of the present invention can be manufactured by known methods, by adding ingredients other than dried Kudzu flower powder according to need. Examples of other ingredients include, for example, vitamins such as water soluble vitamins (vitamin B1, B2, B3, B5, B6, B12, B13, B15, B17, biotin, choline, folic acid, inositol, PABA, vitamin C, vitamin P) and oil soluble vitamins (vitamin A, D, E, K), etc.; minerals such as magnesium, phosphorous, zinc, iron, etc.; sulfur-containing compounds contained in taurine, garlic, etc.; flavonoid or flavonoids, such as hesperidin, quercetin, etc.; protein such as collagen, etc.; peptide; amino acid; animal oil and fat; vegetable oil; crashed products or extracts of animals, plants, etc.

Further, particularly, in case of using the food product of the present invention as powder beverage or tea hag beverage, plant material other than dried Kudzu flower powder can be mixed, and examples of plants include, for example, tea leaves such as green tea leaves or black tea leaves, catnip, Hyssop milk Thistle, yarrow, chicory, etc. Examples of these plant materials include dried powder obtained by drying and grounding collected plants. Among these plant materials, by using tea leaves, particularly green tea leaves in combination, better taste can be obtained,

### EXAMPLES

In the following, the present invention will be explained in further details by the Examples. However, the present invention is not limited to these Examples, and the present. invention can have various embodiments as long as it can resolve the object of the present invention.

### 1. Sensory Test

### 1-1. Comparison by Water Content

### <Preparation of Dried Kudzu Flower Powder>

Collected flowers of Kudzu were dried by solar drying and hot-air drying. In hot-air drying, drying time was adjusted to each have a predetermined water content. For flowers of Kudzu after drying (dried Kudzu flower), grounding condition was adjusted so that the median diameter (weight basis) is about 0.4 mm, proportion of fractions passing a sieve having a sieve opening of 2 mm is 90% or more, proportion of fractions passing a sieve opening of 2 mm and not passing a sieve having a sieve opening of 0.25 mm is 70 to 81%, to produce dried Kudzu flower powder of Examples 1 to 4 and Comparative Example 1 which median diameter and particle size distribution are comparable. After producing the dried Kudzu flower powder, and storing at ambient temperature (25° C.) for one month or more, the tests were performed. The water content of Examples 1 to 4 and Comparative Example 1 is shown in Table 1.

TABLE 1

| | Water content (%) |
|---|---|
| Example 1 | 2.9% |
| Example 2 | 3.2% |
| Example 3 | 4.7% |
| Example 4 | 7.1% |
| Comparative Example 1 | 8.4% |

### <Implementation of Sensory Test>

For Examples 1 to 4 and Comparative Example 1, sensory test was performed by the methods described in the following, to assess savor, etc.
(1) 1 g of dried Kudzu flower powder in a test cup was ingested by 8 test subjects, to assess items such as strongness of taste, etc. Assessment was made by scoring with the following criteria, by comparison with Comparative Example 1. The results are shown in Table 2.
3 very good
2 good
1 rather good
−0 no change
−1 rather bad
−2 bad
−3 very bad

TABLE 2

| Item assessed | Example 1 | Example 2 | Example 3 | Example 4 |
|---|---|---|---|---|
| Strongness of taste | 0.6 | 1.6 | 1.0 | 0.9 |
| Strongness of flavor | 0.4 | 1.5 | 1.1 | 0.8 |
| Savor | 0.4 | 1.4 | 0.9 | 1.0 |
| Bitterness | 0.1 | 1.1 | 0.6 | 0.9 |
| Richness of tase | 0.1 | 0.9 | 0.9 | 0.3 |
| Palateness (feeling of | 0.4 | 1.0 | 1.1 | 0.6 |
| particle) | | | | |
| Texture※ | 0.3 | 0.6 | 1.1 | 0.9 |

※good when crispy, bad when crumbly

(2) Tea bags filled with dried Kudzu flower powder were extracted with boiling water, were ingested by 6 test subjects, and assessed. Specifically, first, 1.7 g of dried Kudzu flower power was stuffed in tea bags. After pouring 300 mL of boiling water on tea bags, and allowed to rest for 3 minutes, the tea bags were tossed up and down about 10 times, and taken away. About 30 mL of extraction liquid was put into a test cup, and sweetness when ingested, richness, stickiness in mouth, and unpleasantness in mouth after ingestion were assessed. Assessment criteria are the same as the above (1). The results are shown in Table 3.

TABLE 3

| | | Exam- | Exam- | Exam- | Exam- |
|---|---|---|---|---|---|
| Items | | ple | ple 2 | ple 3 | ple 4 |
| At the | Sweetness | 0.2 | 0.7 | 0.5 | 0.3 |
| time of | Richness | 0.7 | 1.2 | 1.2 | 0.8 |
| ingestion | (hearty taste) | | | | |
| After | Stickiness | 0.2 | 0.3 | 0.3 | 0.3 |
| ingestion | in mouth ※ | | | | |
| | Unpleasantness | 0.2 | 0.7 | 0.7 | 0.3 |
| | in mouth | | | | |

※ good when no stickiness is felt, and bad when stickiness is felt

From Tables 2 and 3, it was observed that by adjusting the water content of dried Kudzu flower powder to 8% by weight or less, dried Kudzu flower powder excellent in savor, etc, can be obtained (Examples 1 to 4). Particularly, dried Kudzu flower powder which water content is 3 to 8% by weight, savor, sweetness, etc. were significantly superior (Examples 2 to 4). Further, when using Kudzu flower extract, even the water content was the same, savor or taste was bad compared to dried Kudzu flower powder.

### 1-2. Comparison by Median Diameter (Weight Basis)

### <Preparation of Dried Kudzu Flower Powder>

Collected flowers of Kudzu were dried by solar drying and hot-air drying. In hot-air drying, drying time was adjusted to each have a predetermined water content. Flower of Kudzu after drying (dried Kudzu flower) was subjected to grounding treatment by adjusting the grounding conditions, and by fractioning with sieves, dried Kudzu flower powders of Examples 5 to 8 having a water content of 3.2% by weight and having different median diameter were prepared. Median diameter of Examples 5 to 8 is shown in Table 4.

TABLE 4

| | Median diameter (mm) |
|---|---|
| Example 5 | 0.17 |
| Example 6 | 0.32 |
| Example 7 | 1.55 |
| Example 8 | 1.67 |

### <Implementation of Sensory Test>

5 test subjects took a test cup containing 1 g of dried Kudzu flower powder close to the nose, to assess the sweetness of odor. Then, dried Kudzu flower powder was pinched from the cup and ingested, to assess the aftertaste. After ingestion, odor remaining in the mouth cavity was assessed. Assessment criteria are the same as the above 1-1 (1). The results are shown in Table 5.

TABLE 5

| | | Exam- | Exam- | Exam- | Exam- |
|---|---|---|---|---|---|
| Items | | ple 5 | ple 6 | ple 7 | ple 8 |
| Before | Sweetness | 0.2 | 1.2 | 0.5 | 0.2 |
| ingestion | of odor | | | | |
| At the time | Aftertaste | 0.3 | 1.0 | 1.0 | 0.8 |
| of ingestion | | | | | |
| After ingestion | Odor | 0.5 | 1.2 | 0.7 | 0.5 |

As shown in Table 5, dried Kudzu flower powder with a median diameter of 0.18 mm or more (Examples 6-8) had a better aftertaste at the time of ingestion as compared to dried Kudzu flower powder with a median diameter of 0.17 mm (Example 5). Particularly, dried Kudzu flower powder with a median diameter of 0.18 to 1.65 mm (Examples 6 and 7) was further superior in sweetness of odor before ingestion, and odor after ingestion.

### 1-3. Comparison by Specific Surface Area

### <Preparation of Dried Kudzu Flower Powder>

Collected flowers of Kudzu were dried by solar drying and hot-air drying. In hot-air drying, drying time was adjusted to each have a predetermined water content. Flower of Kudzu after drying (dried Kudzu flower) was subjected to grounding treatment by adjusting the grounding conditions, and by fractioning with sieves, dried Kudzu flower powders of Examples 9 and 10 having a water content of 3.2% by weight and having different specific surface area were prepared. Specific surface area of Examples 9 and 10 is shown in Table 6.

TABLE 6

| | Specific surface area (m^{2/}g) |
|---|---|
| Example 9 | 0.424 |
| Example 10 | 0.719 |

### <Implementation of Sensory Test>

5 test subjects took a test cup containing 1 g of dried Kudzu flower powder close to the nose, to assess the sweetness of odor. Then, dried Kudzu flower powder was pinched from the cup and ingested, to assess the savor, richness of taste, and aftertaste. After ingestion, odor remaining in the mouth cavity was assessed. Assessment criteria are the same as the above 1-1 (1). The results are shown in Table 7.

| Items | | Example 9 | Example 10 |
|---|---|---|---|
| Before ingestion | Sweetness of odor | 0.2 | 0.5 |
| At the time of | Savor | 1.0 | 1.2 |
| ingestion | Richness of taste | 0.7 | 1.3 |
| | Aftertaste | 0.3 | 1.0 |
| After ingestion | Odor | 0.5 | 0.7 |

As shown in Table 7, dried Kudzu flower powder with a specific surface area of 0.5 m²/g or more (Example 10) was superior in sweetness of odor before ingestion, savor, richness of taste and aftertaste at the time of ingestion, and odor after ingestion as compared to dried Kudzu flower powder with a specific surface area of less than 0.5 m²/g (Example 9).

### 1-4. Comparison by Particle Size Distribution

### <Preparation of Dried Kudzu Flower Powder>

Collected flowers of Kudzu were dried by solar drying and hot-air drying. In hot-air drying, drying time was adjusted to each have a predetermined water content. Flower of Kudzu after drying (dried Kudzu flower) was subjected to grounding treatment by adjusting the grounding conditions, and by fractioning with sieves, dried Kudzu flower powders of Examples 11 to 13 having a water content of 3.2% by weight and having different particle size distribution were prepared. Particle size distribution and median diameter of Examples 11 to 13 are shown in Table 8.

TABLE 8

| | | | | | | | | | | Fractions | |
|---|---|---|---|---|---|---|---|---|---|---|---|
| | | | | | | | | | | of 2 mm | |
| | Particle size distribution (% by weight of on product at each sieve opening) | | | | | | | | | pass and | Median |
| | <0.053 | 0.053 | 0.09 | 0.15 | 0.25 | 0.425 | 0.71 | 1.18 | 2 | 0.25 mm | diameter |
| | mm | mm | mm | mm | mm | mm | mm | mm | mm | on | mm |
| Example 11 | 3.82 | 11.13 | 23.21 | 56.27 | 5.09 | 0.32 | 0.16 | 0 | 0 | 5.57 | 0.17 |
| Example 12 | 2.66 | 9.61 | 18.81 | 45.2 | 21.47 | 2.25 | 0 | 0 | 0 | 23.72 | 0.19 |
| Example 13 | 0 | 0 | 0.14 | 0.28 | 0.28 | 0.7 | 4.64 | 77.5 | 16.46 | 83.12 | 1.65 |

### <Implementation of Sensory Test>

5 test subjects took a test cup containing 1 g of dried Kudzu flower powder close to the nose, to assess the sweetness of odor. Then, dried Kudzu flower powder was pinched from the cup and ingested after assessing the darkness of color. After ingestion, odor remaining in the mouth cavity was assessed. Assessment criteria are the same as the above 1-1 (1). The results are shown in Table 9.

TABLE 9

| | | Exam- | Exam- | Exam- |
|---|---|---|---|---|
| Items | | ple 11 | ple 12 | ple 13 |
| Before ingestion | Sweetness of odor | 0.2 | 0.5 | 0.3 |
| At the time of ingestion | Darkness of color | 0.7 | 1.2 | 1.0 |
| After ingestion | Odor | 0.5 | 1.3 | 0.7 |

As shown in Table 9, dried Kudzu flower powder (Examples 12 and 13) which proportion of fractions passing a sieve opening of 2 mm and not passing a sieve opening of 0.25 mm is 20% by weight or more was superior in sweetness of odor before ingestion, darkness of color at the time of ingestion, and odor after ingestion as compared to those which proportion of fraction is less than 20% by weight (Example 11).

### 1-5. Comparison by Repose Angle

### <Preparation of Dried Kudzu Flower Powder>

Collected flowers of Kudzu were dried by solar drying and hot-air drying. In hot-air drying, drying time was adjusted to each have a predetermined water content. Flower of Kudzu after drying (dried Kudzu flower) was subjected to grounding treatment by adjusting the grounding conditions, and by fractioning with sieves, dried Kudzu flower powders of Examples 14 to 17 having a water content of 3.2% by weight and having different repose angle were prepared. Repose angle of Examples 14 to 17 is shown in Table 10.

TABLE 10

| | Repose angle (°) |
|---|---|
| Example 14 | 54 |
| Example 15 | 44 |
| Example 16 | 51 |
| Example 17 | 47 |

### <Implementation of Sensory Test>

5 test subjects took a test cup containing 1 g of dried Kudzu flower powder close to the nose, to assess the sweetness of odor. Then, dried Kudzu flower powder was pinched from the cup and ingested after assessing darkness of color. After ingestion, odor remaining in the mouth cavity was assessed. Assessment criteria are the same as the above 1-1 (1). The results are shown in Table 11.

TABLE 11

| | | Exam- | Exam- | Exam- | Exam- |
|---|---|---|---|---|---|
| Items | | ple 14 | ple 15 | ple 16 | ple 17 |
| Before | Sweetness | 0.2 | 0.2 | 0.5 | 0.3 |
| ingestion | of odor | | | | |
| At the time | Darkness | 0.7 | 0.8 | 1.2 | 1.0 |
| of ingestion | of color | | | | |
| After ingestion | Odor | 0.5 | 0.5 | 1.3 | 0.7 |

As shown in Table 11, dried Kudzu flower powder with a repose angle of 53 degree or less (Examples 15 to 17) was superior in darkness of color at the time of ingestion as compared to dried Kudzu flower powder with a repose angle of 54 degree (Example 14). Further; dried Kudzu flower powder with a repose angle of 45 degree or more (Examples 16 and 17) was superior in sweetness of odor before ingestion and odor after ingestion as compared to dried Kudzu flower powder with a repose angle of 44 degree (Example 15).

### 2. Elution Test

### 2-1. Comparison by Water Content (1)

### <Preparation of Dried Kudzu Flower Powder>

Collected flowers of Kudzu were dried by solar drying and hot-air drying. In hot-air drying, drying time was adjusted to each have a predetermined water content. For flower of Kudzu after drying (dried Kudzu flower), grounding condition was adjusted so that the median diameter (weight basis) is about 0.4 mm, proportion of fractions passing a sieve opening of 2 mm is 90% or more, proportion of fractions passing a sieve opening of 2 mm and not passing a sieve opening of 0.2.5 mm is 70 to 81%, to produce dried Kudzu flower powders of Examples 18 and 19 and Comparative Example 2 which median diameter and particle size distribution are comparable. After producing the dried Kudzu flower powders, and storing at ambient temperature (25° C.) for one month or more, the tests were performed. The water content of Examples 18 and 19 and Comparative Example 2 is shown in Table 12.

TABLE 12

| | Water content (%) |
|---|---|
| Example 18 | 4.7% |
| Example 19 | 7.1% |
| Comparative Example 2 | 8.4% |

### <Implementation of Elution Test>

100 mg of dried Kudzu flower powder was measured out in a 1.5 mL tube, added with 1 mL of pure water, and vortexed for 30 sec. Absorbance at 465 nm of the supernatant after centrifuge was measured. Absorbance of pure water was measured as blank. For the value from which the level of blank was subtracted, using Comparative 2 as 100, the values of Examples 18 and 19 were assessed as absorbance. Higher is the absorbance, it shows that the eluting property of ingredients contained in dried Kudzu flower powder is high. The results are shown in Table 13.

TABLE 13

| | Absorbance |
|---|---|
| Example 18 | 163.73 |
| Example 19 | 117.96 |
| Comparative Example 2 | 100 |

Dried Kudzu flower powder which water content is 8% by weight or less (Examples 18 and 19) showed a high level of absorbance, as compared to dried Kudzu flower powder which water content is greater than 8% by weight (Comparative Example 2), and the eluting property of ingredients contained in dried Kudzu flower powder was superior.

### 2-2. Comparison by Water Content (2)

### <Preparation of Dried Kudzu Flower Powder>

Collected flowers of Kudzu were dried by solar drying and hot-air drying. In hot-air drying, drying time was adjusted to each have a predetermined water content. For flower of Kudzu after drying (dried Kudzu flower), grounding condition was adjusted so that the median diameter (weight basis) is about 0.4 mm, proportion of fractions passing a sieve opening of 2 mm is 90% or more, proportion of fractions passing a sieve opening of 2 mm and not passing a sieve opening of 0.2.5 mm is 70 to 81%, to produce dried Kudzu flower powders of Examples 20 and 21 which median diameter and particle size distribution are comparable. After producing the dried Kudzu flower powders, and storing at ambient temperature (25° C.) for one month or more, the tests were performed. The water content of Examples 20 and 21 is shown in Table 14.

TABLE 14

| | Water content (%) |
|---|---|
| Example 20 | 3.2% |
| Example 21 | 2.9% |

### <Implementation of Elution Test>

100 mg of dried Kudzu flower powder was measured out in a 1.5 mL tube, added with 1 mL of pure water, and vortexed for 30 sec. Absorbance at 465 nm of the supernatant after centrifuge was measured. Absorbance of pure water was measured as blank. For the value from which the level of blank was subtracted, using Example 21 as 100, the value of Example 20 was assessed as absorbance. The results are shown in Table 15.

TABLE 15

| | Absorbance |
|---|---|
| Example 20 | 110.92 |
| Example 21 | 100 |

Dried Kudzu flower powder which water content is 3% by weight or more (Example 20) showed a high level of absorbance, as compared to dried Kudzu flower powder which water content is less than 3% by weight (Example 21), and the eluting property of ingredients contained in dried Kudzu flower powder was superior.

### 2-3. Comparison by Median Diameter

### <Preparation of Dried Kudzu Flower Powder>

Collected flowers of Kudzu were dried by solar drying and hot-air drying. In hot-air drying, drying time was adjusted to each have a predetermined water content. Flower of Kudzu after drying (dried Kudzu flower) was subjected to grounding treatment by adjusting the grounding conditions, and by fractioning with sieves, dried Kudzu flower powders of Examples 22 to 26 having a water content of 3,2% by weight and having different median diameter were prepared. Median diameter of Examples 22 to 26 is shown in Table 16.

TABLE 16

| | Median diameter (mm) |
|---|---|
| Example 22 | 0.19 |
| Example 23 | 0.32 |
| Example 24 | 1.55 |
| Example 25 | 1.65 |
| Example 26 | 1.67 |

### <Implementation of Elution Test>

100 mg of dried Kudzu flower powder was measured out in a 1.5 mL tube, added with 1 mL of pure water, and vortexed for 30 sec. Absorbance at 465 nm of the supernatant after centrifuge was measured. Absorbance of pure water was measured as blank. For the value from which the level of blank was subtracted, using Example 26 as 100, the values of Examples 22 to 25 were assessed as absorbance. The results are shown in Table 17.

TABLE 1

| | Asorbance |
|---|---|
| Example 22 | 249.73 |
| Example 23 | 125.54 |
| Example 24 | 302.12 |
| Example 25 | 136.32 |
| Example 26 | 100 |

Dried Kudzu flower powder with median diameter 1.65 mm or less (Examples 22 to 25) showed a high level of absorbance, as compared to dried Kudzu flower powder with median diameter 1.67 mm (Example 26), and the eluting property of ingredients contained in dried Kudzu flower powder was superior.

### 3. Analysis of Tectorigenins

By the method described in the following, the amount of tectorigenins contained in dried Kudzu flower powder (total amount of tectorigenin, tectoridin, and tectorigenin 7-O-xylosylglucoside) was analyzed. In dried Kudzu flower powder of Examples 1 to 26, content of tectorigenins was 5 to 15% by weight.

Measurement of tectorigenins was measured by HPLC, by refluxing dried Kudzu flower powder with 50% methanol. Specifically, as analysis column, YMC—Pack ODS AM12S05-2546WT (φ4.6×250 mm) manufactured by YMC CO., LTD.; as guard column, YMC-Pack ODS AM12S05-G304CC (φ4.0×23 mm) or YMC-Pack ODS AM1250S-0204GC (φ4.0×20 mm) manufactured by YMC CO., LTD. was used; as liquid medium of mobile phase, acetonitrile/water/acetic acid mixed liquid (mobile phase A volume ratio=15:85:0.1, mobile phase B volume ratio=35:65:0.1) was used, and the column temperature was 35° C., and flow rate was 1.0 ml/min.

The gradient conditions are as described in Table 18.

TABLE 18

| Time (min) | A liquid (%) | B liquid (%) |
|---|---|---|
| 0 | 100 | 0 |
| 50 | 0 | 100 |
| 55 | 0 | 100 |
| 57 | 100 | 0 |
| 70 | 100 | 0 |

4. Comparison with Kudzu Flower Extract

### <Preparation of Dried Kudzu Flower Powder>

Collected flowers of Kudzu were dried by solar drying and hot-air drying so that water content becomes 5%. Dried Kudzu flower was grounded, and dried Kudzu flower powder with median diameter of about 0.4 mm was used for test.

On the other hand, collected flowers of Kudzu was extracted with hot water, filtered, and dried by hot-air drying so that the water content becomes 5%.

### <Implementation of Sensory Test>

For the above-mentioned dried Kudzu flower powder and Kudzu flower extract powder, sensory test was performed by the two following methods.
(1) 4 test subjects took a test cup containing 1 g of dried Kudzu flower, to assess the items such as sweetness of odor before ingestion. Assessment was made by scoring with the following criteria according to the comparison with the Kudzu flower extract powder. The results are shown in Table 19.
3 very good
2 good
1 rather good
0 no change
−1 rather had
−2 bad
−3 very bad

TABLE 19

| | | |
|---|---|---|
| Before ingestion | Sweetness of odor | 2.5 |
| At the time of | Strongness of odor | 2.3 |
| ingestion | Savor | 2.0 |
| | Bitterness | 2.5 |
| | Richness of taste | 1.5 |
| | Aftertaste | 3.0 |
| After ingestion | Odor | 2.5 |

As shown in Table 19, in each item assessed, dried Kudzu flower powder was superior as compared to Kudzu flower extract powder.
(2) Tea bags filled with dried Kudzu flower powder were extracted with boiling water, and were ingested and assessed by 4 test subjects. Specifically, first, 1.7 g of dried Kudzu flower power was stuffed in tea bags. After pouring 300 mL of boiling water on tea bags, and allowed to rest for 3 minutes, the tea bags were tossed up and down about 10 times, and taken away. Extraction liquid was put in a test cup, and sweetness and richness when ingested, stickiness in mouth and unpleasantness in mouth after ingestion were assessed. Assessment criteria are the same as the above (1). The results are shown in Table 20.

TABLE 20

| | | |
|---|---|---|
| At the time of | Sweetness | 2.8 |
| ingestion | Richness (hearty taste) | 2.3 |
| After ingestion | Stickiness in mouth | 2.3 |
| | Unpleasantness in mouth | 3.0 |

As shown in Table 20, in each item to be assessed, dried Kudzu flower powder was superior as compared to Kudzu flower extract powder.

### 5. Elution Test and Cell Activation Test

### 5-1. Comparison by Water Content

### <Preparation of Dried Kudzu Flower Powder>

Collected flowers of Kudzu were dried by solar drying and hot-air drying. In hot-air drying, drying time was adjusted to each have a predetermined water content. For flowers of Kudzu after drying (dried Kudzu flower), grounding condition was adjusted so that the median diameter (weight basis) is about 0.4 mm, proportion of fractions passing a sieve having a sieve opening of 2 mm is 90% or more, proportion of fractions passing a sieve having a sieve opening of 2 mm and not passing a sieve having a sieve opening of 0.25 mm is about 70 to 80%, to produce dried Kudzu flower powders of Examples and Comparative Examples which median diameter and particle size distribution are comparable. After producing the dried Kudzu flower powders, and storing at ambient temperature (25° C.) for one month or more, the tests were performed. The water content of Examples 27 to 30 and Comparative Example 3 is shown in Table 21.

TABLE 21

| | Water content (%) |
|---|---|
| Example 27 | 2.4 |
| Example 28 | 3.2 |
| Example 29 | 4.7 |
| Example 30 | 7.8 |
| Comparative Example 3 | 8.4 |

### <Implementation of Elution Test>

Elution test was performed by the following method.
(1) About 200 mg of dried Kudzu flower powder was weighed in a 5 mL-sampling tube.
(2) Serum free-DMEM was added to be 100 mg/mL, and the mixture was stirred with a tube mixer for 2 min.
(3) The resultant was centrifuged at room temperature at 15000×g for 2 min., and supernatant was collected in a 1.5 mL-sampling tube.
(4) To remove fine powder, the mixture was further centrifuged (room temperature, 15000×g for 2 min.) to collect supernatant.
(5) Supernatant was diluted 100-fold with serum free-DMEM, aliquoted in an assay plate (UV-Star) 100 L each, to measure the absorbance at 264 nm. Further, as blank, the absorbance of only serum free-DMEM was similarly measured to amend the absorbance.

The results are shown in Table 22. The elution property is shown by a relative value using the absorbance after blank correction of Comparative Example 3 at 264 nm as 100. Greater is the level, it shows that the elution property is high.

TABLE 22

| | Absorbance (relative value) |
|---|---|
| Example 27 | 136.6 |
| Example 28 | 133.2 |
| Example 29 | 124.3 |
| Example 30 | 119.4 |
| Comparative Example 3 | 100 |

Dried Kudzu flower powder which water content is 8% by weight or less (Examples 27 and 30) was superior in eluting property, as compared to dried Kudzu flower powder which water content is greater than 8% by weight (Comparative Example 3).

### <Implementation of Cell Activation Test>

Cell activation test was performed by the following method.
(1) Caco-2 cells cultured in 10% FBS-1% NEAA-DMEM medium were seeded in 96 well plate, and precultured in CO₂ incubator at 37° C.
(2) After preculture, the resultant was washed once with serum free-DMEM.
(3) The sample (100 mg/mL) eluted in the above (4) of <Implementation of elution test> was diluted to 1.25 mg/mL with serum free-DMEM, and added to each well. As control, serum free-DNIEM medium was added.
(4) After adding the sample, the resultant was cultured in CO₂ incubator at 37° C. for 6 hours.
(5) The resultant was washed once with serum-free DMEM medium, and Cell Counting Kit-8 diluted 30-fold with serum free-DMEM was added 150 μL/well. As blank, it was similarly added to well without cell.
(6) After appropriately forming color in CO₂ incubator at 37° C., absorbance at 450 nm was measured. Correcting with the absorbance of blank, cell activation was calculated.

The results are shown in Table 23. The cell activation was shown by a relative value, using control as 100, and greater is the level, it shows that the cell activation is high.

TABLE 23

| | Cell activation (relative value) |
|---|---|
| Control | 100 |
| Example 27 | 105.1 |
| Example 28 | 110.6 |
| Example 29 | 111.6 |
| Example 30 | 112.1 |

For all Examples, the level of cell activation was superior as compared to control. As compared to Example 27 which water content is less than 3%, Examples 28 to 30 which water content is 3% or more and 8% or less, the cell activation was high.

### 5-2. Comparison by Median Diameter

### <Preparation of Dried Kudzu Flower Powder>

Collected flowers of Kudzu were dried by solar drying and hot-air drying. In hot-air drying, drying time was adjusted to each have a predetermined water content. Flower of Kudzu after drying (dried Kudzu flower) was subjected to grounding treatment by adjusting the grounding conditions, and by fractioning with sieves, dried Kudzu flower powders of Examples 31 to 34 having a water content of 3.2% by weight, and having different median diameter were prepared. Median diameter of Examples 31 to 34 is shown in Table 24.

TABLE 24

| | Median diameter (mm) |
|---|---|
| Example 31 | 0.17 |
| Example 32 | 0.32 |
| Example 33 | 1.55 |
| Example 34 | 1.67 |

### <Implementation of Dissolution Test>

Test was performed similarly as the elution test in the above “5-1. Comparison by water content”. In the present test, in (1), about 150 mg of dried Kudzu flower powder was weighed, and in (2) it was stirred for 10 min. with a tube mixer.

The results are shown in Table 25. The elution property is shown by a relative value using the absorbance after blank correction of Example 34 at 264 nm as 100. Greater is the level, it shows that the elution property is high.

TABLE 25

| | Absorbance (relative value) |
|---|---|
| Example 31 | 283.5 |
| Example 32 | 239.6 |
| Example 33 | 131.8 |
| Example 34 | 100 |

As compared to Example 34 which median diameter is greater than 1.65 mm, dried Kudzu flower powder of Examples 31 to 33 which median diameter is 1.65 nm or less was superior in eluting property.

### <Implementation of Cell Activation Test>

Test was performed similarly as the elution test in the above “5-1. Comparison by water content”. In the present test, in (2), eluted sample was diluted to 3 mg/mL, and in (4), the resultant was cultured for 8 hours.

The results are shown in Table 26. The cell activation was shown by a relative value, using control as 100, and greater is the level, it shows that the cell activation is high.

TABLE 26

| | Cell activation (relative value) |
|---|---|
| Control | 100 |
| Example 31 | 106.6 |
| Example 32 | 121.8 |
| Example 33 | 121.6 |
| Example 34 | 122.5 |

For all Examples, the level of cell activation was superior as compared to control. As compared to Example 31 which median diameter is less than 0.17 mm, Examples 32 to 34 which median diameter is 0.18 mm or more, the cell activation was higher.

### 6. Confirmation of Change in Cholesterol in Blood by Ingestion of Dried Kudzu Flower Powder

### <Preparation of Dried Kudzu Flower Powder>

Collected flowers of Kudzu were dried by solar drying and hot-air drying. In hot-air drying, drying time was adjusted to each have a predetermined water content. Flower of Kudzu after drying (dried Kudzu flower) was subjected to grounding treatment by adjusting the grounding conditions, and by fractioning with sieves, dried Kudzu flower powder having comparable water content (about 5% by weight), and having different median diameter (1.75 Mm, 0.45 mm) were prepared.

### <Implementation of Animal Test>

After acclimating male C57BL/6J mice, mice were divided in groups according to their body weight, and allowed to freely ingest feed shown in the following Table 27 for 36 days, in each group. On the last day of test, mice were dissected, blood was collected front postcaval vein, to obtain serum. By using serum, and using kits available on market (product name “cholesterol E-test WAKO”; manufactured by FUJIFILM Wako Pure Chemical Corporation), total cholesterol level in blood was measured. The results are shown in Table 28.

TABLE 27

| | Control | Example 35 | Example 36 |
|---|---|---|---|
| Milk casein | 20 | 20 | 20 |
| α potato starch | 28.2 | 21.26 | 21.26 |
| Sucrose | 13 | 13 | 13 |
| Corn oil | 20 | 20 | 20 |
| Lard | 10 | 10 | 10 |
| Cellulose | 4 | 4 | 4 |
| Mineral mixture AIN-76 | 3.5 | 3.5 | 3.5 |
| Vitamin mixture AIN-76 | 1 | 1 | 1 |
| DL-methionine | 0.3 | 0.3 | 0.3 |
| Dried Kudzu flower powder | | 6.94 | |
| median diameter 1.75 mm | | | |
| Dried Kudzu flower powder | | | 6.94 |
| median diameter 0.45 mm | | | |
| Total | 100 | 100 | 100 |

TABLE 28

| | | Total cholesterol |
|---|---|---|
| | | level in blood |
| | Feed | (mg/dL)* |
| Control | Only high-fat diet | 131.7 |
| Example 35 | High-fat diet + dried Kudzu | 125.7 |
| | flower powder | |
| Example 36 | High-fat diet + dried Kudzu | 116.9 |
| | flower powder | |

By ingesting dried Kudzu flower powder, total cholesterol level in blood decreased. Particularly, in Example 36 with a smaller particle diameter, a large decrease was observed.

7. Sensory Test with the Combination of Dried Kudzu Flower Powder And Green Tea

### <Implementation of Sensory Test>

Tea bags filled with dried Kudzu flower powder having a predetermined water content and median diameter and dried green tea powder were extracted with boiling water, and were ingested and assessed by 5 test subjects. Specifically, first, 0.45 g of dried Kudzu flower powder and 1.25 g of dried green tea powder were stuffed in tea bags. After pouring 150 mL of boiling water on tea bags, and allowed to rest for 3 minutes, the tea bags were tossed up and down about 10 times, and taken away. Pleasant taste at the time of ingestion, etc. was assessed. For Examples 38 to 40 and Comparative Example 4, composition ratio of dried Kudzu flower powder and dried green tea powder in tea bags, and median diameter and water content of dried Kudzu flower powder used in each Example and Comparative Example are shown in Table 29.

TABLE 29

| | | Example | Example | Example | Example | Comparative |
|---|---|---|---|---|---|---|
| | | 37 | 38 | 39 | 40 | Example 4 |
| Tea | Dried Kudzu flower powder | 26% | | | | |
| bag | (median diameter 0.17 mm, water | | | | | |
| | content 3.2%) | | | | | |
| | Dried Kudzu flower powder (median | | 26% | | | |
| | diameter 0.32 mm, water content 3.2%) | | | | | |
| | Dried Kudzu flower powder (median | | | 26% | | |
| | diameter 1.55 mm, water content 3.2%) | | | | | |
| | Dried Kudzu flower powder (median | | | | 26% | |
| | diameter 1.67 mm, water content 3.2%) | | | | | |
| | Dried Kudzu flower powder (median | | | | | 26% |
| | diameter 0.44 mm, water content 8.4%) | | | | | |
| | Green tea dried powder | 74% | 74% | 74% | 74% | 74% |
| | Total | 100% | 100% | 100% | 100% | 100% |

Assessment was made by scoring with the following criteria, by comparison with Comparative Example 4. The results are shown in Table 30.
3 very good
2 good
1 rather good
0 no change
−1 rather bad
−2 bad
−3 very bad

TABLE 30

| Item | Example 37 | Example 38 | Example 39 | Example 40 |
|---|---|---|---|---|
| Pleasant taste | 1.2 | 1.8 | 1.8 | 1.0 |
| Harsh taste | 1.0 | 2.2 | 1.8 | 1.0 |
| Richness of taste | 1.2 | 1.6 | 2.0 | 1.0 |
| Aftertaste | 1.2 | 2.2 | 2.0 | 1.0 |
| Mouthfeel | 0.8 | 2.2 | 1.8 | 1.4 |

As shown in Table 30, in the combination of dried Kudzu flower powder and dried green tea powder, by using dried Kudzu flower powder having a water content of 8% or less of the present invention, good results were obtained. Further, in the combination of dried Kudzu flower powder with median diameter of 0.18 to 1.65 mm and dried green tea powder (Examples 38 and 39), superior results were obtained.

### Blending Example 1

Tea bags (2 g per bag) comprising dried Kudzu flower powder were produced. Dried Kudzu flower powder had a water content of 4% by weight, and median diameter (weight basis) of 1 mm. Beverage using the obtained tea bags had good taste and odor. Further, by ingesting this beverage once a day, excellent body fat reducing effect etc. can be obtained.

### Blending Example 2

Tea bags (2 g per bag) comprising dried Kudzu flower powder (70% by weight) and dried catnip powder (30% by weight) were produced. Dried Kudzu flower powder had a water content of 4% by weight, and median diameter (weight basis) of 1 mm. Beverage using the obtained tea bags had good taste and odor. Further, by ingesting this beverage once a day, excellent body fat reducing effect etc. can be obtained.

### Blending Example 3

Tea bags (2 g per bag) comprising dried Kudzu flower powder (50% by weight) and dried hyssop powder (50% by weight) were produced. Dried Kudzu flower powder had a water content of 6% by weight, and median diameter (weight basis) of 1.4 mm. Beverage using the obtained tea bags had good taste and odor. Further, by ingesting this beverage once a day, excellent body fat reducing effect etc. can be obtained.

### Blending Example 4

Food product (2 g) comprising dried Kudzu flower powder (70% by weight) and dried catnip powder (30% by weight) were produced. Dried Kudzu flower powder had a water content of 4% by weight, and median diameter (weight basis) of 1 mm. Obtained food products had good taste and odor. Further, by ingesting this food product once a day, excellent body fat reducing effect etc. can be obtained.

### INDUSTRIAL APPLICABILITY

Since the food product of the present invention can be used as general food product as well as health food products, etc. it has a high industrial applicability.

1. A dried Kudzu flower powder, having a water content of 8% by weight or less.

2. The dried Kudzu flower powder according to claim 1, wherein a proportion of fractions passing a sieve having a sieve opening of 2 mm is 20% by weight or more.

3. A food product comprising the dried Kudzu flower powder according to claim 1.

4. A food product comprising a dried Kudzu flower powder, and having a water content of 8% by weight or less.

5. A food product comprising the dried Kudzu flower powder according to claim 2.
